(19) United States
(12) Patent Application Publication
(10) Pub. No.: US 2025/0116659 A1
(43) Pub. Date: Apr. 10, 2025

(54) METAMATERIAL SENSING PLATFORM BASED ON NANODISK STRUCTURE

(71) Applicant: KOREA INSTITUTE OF SCIENCE AND TECHNOLOGY, Seoul (KR)

(72) Inventors: Minah Seo, Seoul (KR); Hyun Seok Song, Seoul (KR); Taehee Kang, Seoul (KR); Taeyeon Kim, Seoul (KR); Yeeun Roh, Seoul (KR); Geon Lee, Seoul (KR); Yeonkyung Lee, Seoul (KR); Jisung Kwak, Seoul (KR)

(21) Appl. No.: 18/781,520

(22) Filed: Jul. 23, 2024

(30) Foreign Application Priority Data
Oct. 6, 2023 (KR) 10-2023-0133512
Oct. 16, 2023 (KR) 10-2023-0137936

(51) Int. Cl. G01N 33/50; G01N 33/531; G01N 33/68; G02B 1/00

(52) U.S. Cl. CPC G01N 33/5091; G01N 33/531; G01N 33/6872; G02B 1/002; G01N 2500/00

(57) ABSTRACT

Disclosed herein is a metamaterial sensing platform. The metamaterial sensing platform includes: a sensing portion configured to capture a particle; and a terahertz sensor portion configured to sense the particle by allowing a terahertz electromagnetic wave to be incident on the sensing portion; wherein the sensing portion includes a base substrate and a particle capture portion disposed on the base substrate and having a plurality of slits formed therein through which the particle is captured, and wherein a nanodisk reactive with the particle is disposed in the plurality of slits.

### CROSS REFERENCE TO RELATED APPLICATIONS

The present application claims priorities to Korean Patent Application No. 10-2023-0133512, filed on Oct. 6, 2023 and No. 10-2023-0137936, filed on Oct. 16, 2023, the entire contents of which are incorporated herein for all purposes by this reference.

### BACKGROUND OF THE INVENTION

### Field of the Invention

The present invention relates to a metamaterial sensing platform, and specifically, to a metamaterial sensing platform that is capable of detecting particles of a small molecular material by introducing a nanodisk structure using a bioprotein as a receptor protein for a target material to apply a resonance frequency in a terahertz band.

### Description about National Research and Development Support

This study was supported by the technology development programs of Ministry of Science and ICT, Republic of Korea (Projects No. 1711196504 and No. 1711174963) under the Korea Institute of Science and Technology.

### Documents of Related Art

While most diseases in the modern society are treatable with the advancement of diagnostic and medical technologies, there is still a need for countermeasures against unconquered diseases such as cancer and diseases whose mechanisms are not clearly understood, such as depression and dementia.

For example, when using small molecular material markers for cancer, real-time diagnosis is possible at all times using exhaled breath in daily life through non-invasive diagnostic methods in contrast to conventional biopsy, endoscopy, and the like, but the commercialization is difficult due to the lack of sensor technology that can capture and detect small molecular materials with appropriate performance.

In addition, there are various small molecule biomarkers that are presumed to be involved in the development of depression, but there is a lack of technology to accurately detect and quantify these biomarkers.

Technologies such as electrochemical or immunoreactive biosensors and the like, which have been conventionally used, require expensive instruments and disposable test papers, or simply have a one-time diagnostic effect due to the use of irreversible reactions.

In particular, when it comes to an electrochemical blood glucose sensor, there is a problem that blood drawing is necessarily involved, and when it comes to an immune response biosensor, there is a lack of diagnostic capability on small molecular materials that are attracting attention as disease biomarkers in the clinical field because the immune response biosensor is only applicable to large materials at a protein level.

### SUMMARY OF THE INVENTION

Therefore, the present invention is directed to providing a novel sensing platform capable of capturing and detecting a small molecular material.

In order to achieve the above-described technical objects, there is provided a metamaterial sensing platform according to the present invention. The metamaterial sensing platform may include: a sensing portion configured to capture a particle; and a terahertz sensor portion configured to sense the particle by allowing a terahertz electromagnetic wave to be incident on the sensing portion; in which the sensing portion includes a base substrate and a particle capture portion disposed on the base substrate and having a plurality of slits formed therein through which the particle is captured, and in which a nanodisk reactive with the particle is disposed in the plurality of slits.

In addition, the base substrate according to the present invention may be a Si layer, and a SiO₂ layer that oxidizes the Si layer may be further disposed between the Si layer and the particle capture portion.

In addition, an amine group may be interposed in the nanodisk according to the present invention and the nanodisk may be attached to the SiO₂ layer.

In addition, the particle capture portion according to the present invention may be an Au layer, the Au layer may have a thickness of 150 nm, and the nanodisk may be disposed at a height of 30 nm or less from a surface of the SiO₂.

In addition, the nanodisk according to the present invention may include G protein-coupled receptors (GPCRs), and the nanodisk may be assembled by mixing the G protein-coupled receptors produced from E. coli cells with a membrane support protein produced from E. coli cells.

In addition, the plurality of slits may be formed by recessedly etching the particle capture portion by a photolithography method such that a surface of the SiO₂ layer is exposed.

In addition, the terahertz sensor portion according to the present invention may sense the particle by allowing the terahertz electromagnetic wave to be incident on the sensing portion and detecting the terahertz electromagnetic wave reflected from the sensing portion.

In addition, the particle may be sensed by measuring reflectivity and frequency shift between the terahertz electromagnetic wave being incident on the sensing portion according to the present invention and the terahertz electromagnetic wave being reflected from the sensing portion.

The present invention may provide a metamaterial sensing platform with high sensitivity that gives selectivity of a material and a position using a nanodisk structure.

In addition, the present invention may provide a metamaterial sensing platform that is capable of qualitative and quantitative analysis of a particle to be sensed non-destructively, without dependence on a marked particle, using a spectroscopy based on terahertz frequency.

### BRIEF DESCRIPTION OF THE DRAWINGS

FIG. 1 is a conceptual view schematically illustrating a metamaterial sensing platform according to one embodiment of the present invention.

FIG. 2 is a conceptual view schematically illustrating a particle capture portion of the metamaterial sensing platform according to one embodiment of the present invention.

FIG. 3A and FIG. 3B are conceptual views schematically illustrating states made before and after a particle to be sensed is captured in the particle capture portion of the metamaterial sensing platform according to one embodiment of the present invention.

FIG. 4 is a conceptual view schematically illustrating a nanodisk of the metamaterial sensing platform according to one embodiment of the present invention.

FIG. 5 is a chemical formula schematically illustrating the bonding of an amine group to a SiO₂ layer of the metamaterial sensing platform according to one embodiment of the present invention.

FIG. 6 is a view schematically illustrating the intensity of an electric field concentrated at a slit in the particle capture portion using the metamaterial sensing platform according to one embodiment of the present invention.

FIG. 7A and FIG. 7B are graphs illustrating a change rate of a signal detected using the metamaterial sensing platform according to one embodiment of the present invention.

FIG. 8 is a graph illustrating an amplification ratio of a detecting signal using the metamaterial sensing platform according to one embodiment of the present invention.

### DETAILED DESCRIPTION OF THE INVENTION

Hereinafter, a metamaterial sensing platform 1 according to one embodiment of the present invention will be described, with reference to the accompanying drawings, through a preferred embodiment of the present invention.

Prior to the description, the constituent elements having the same configurations in the several embodiments will be assigned with the same reference numerals and described only in the representative embodiment, and only the constituent elements, which are different from the constituent elements according to the representative embodiment, will be described in other embodiments.

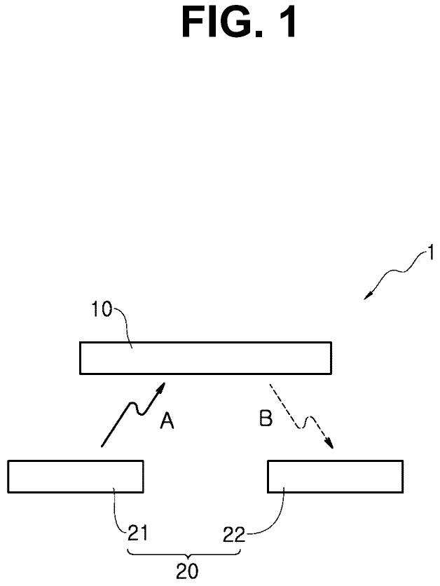

FIG. 1 is a conceptual view schematically illustrating the metamaterial sensing platform 1 according to one embodiment of the present invention.

As illustrated in FIG. 1, the metamaterial sensing platform 1 according to one embodiment of the present invention includes a sensing portion 10 that captures a particle to be sensed, and a terahertz sensor portion 20 that senses the particle to be sensed by allowing a terahertz electromagnetic wave to be incident on the sensing portion 10.

Specifically, the sensing portion 10 comprises a base substrate 100 and a particle capture portion 200 disposed on the base substrate 100 and having a plurality of slits 201 formed therein through which a particle T to be sensed is captured, and a nanodisk 300 reactive with the particle T to be sensed may be disposed in the plurality of slits 201.

The base substrate 100 may be a substrate capable of reflecting the terahertz electromagnetic wave. For example, the base substrate 100 may be a silicon (Si) substrate, but is not limited thereto.

In addition, the particle capture portion 200 formed on the base substrate 100 may be a structure designed to capture the particle T to be sensed and concentrate the terahertz electromagnetic wave on the captured particle. The structure of the particle capture portion 200 may itself be referred to as a metamaterial.

Meanwhile, the terahertz sensor portion 20 is configured to allowing the terahertz electromagnetic wave to be incident on the base substrate 100 of the metamaterial. The terahertz electromagnetic wave may be defined as an electromagnetic wave in the area of 0.1 to 10 THz, with reference to a frequency (1 THz) that oscillates 10¹² times in one second, and the electromagnetic wave in this area has a non-ionizing characteristic and is harmless to the human body when not exceeding a threshold.

As illustrated in FIG. 1, the terahertz sensor portion 20 is positioned on a lower portion of the base substrate 100 of the metamaterial, and may allow the terahertz electromagnetic wave to be incident on one surface of the base substrate 100 from an incident portion 21, and concentrate the terahertz electromagnetic wave reflected from the one surface of the base substrate 100 of the metamaterial to sense the particle T to be sensed captured in the metamaterial 10 at a detector 22.

That is, using the electromagnetic wave in the terahertz band applied to the metamaterial sensing platform 1 according to one embodiment of the present invention, a hormone biomarker, which is a small molecular material, may be experimentally observed through reflectance and frequency shift with the measurement of a reflection signal value detected by an effect in which the particle T to be sensed is captured by the nanodisk 300.

Therefore, since the electromagnetic wave in the terahertz band does not pass through the material to be sensed, but rather measures the reflection signal value that is reflected back, a sensor platform may be provided that is capable of detecting the material to be sensed with no damage to the material.

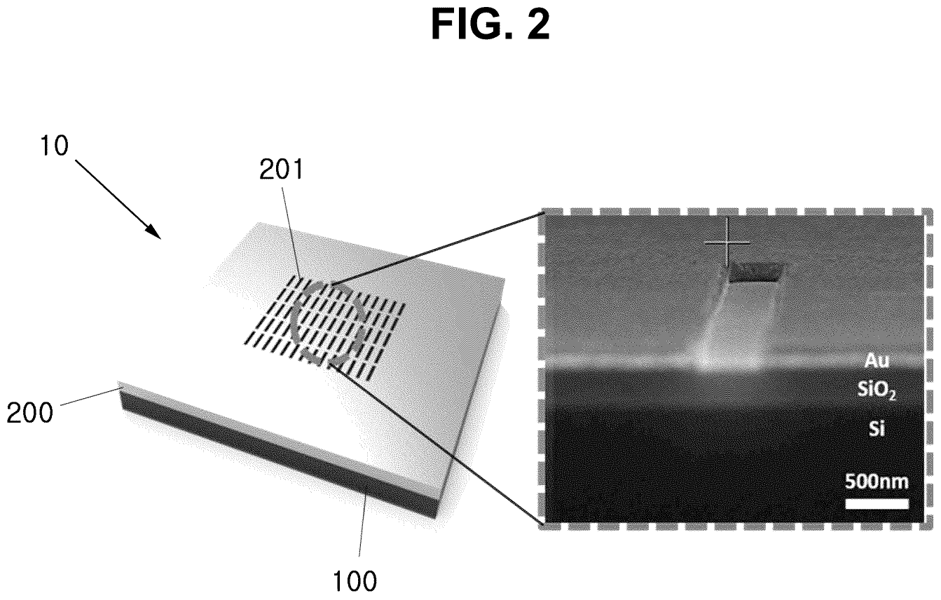

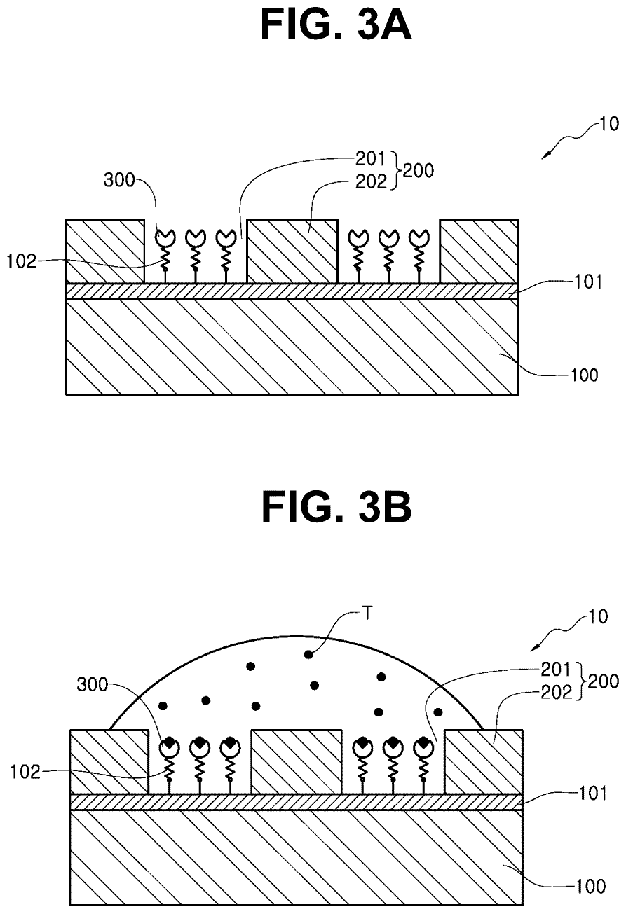

FIG. 2 is a conceptual view schematically illustrating the particle capture portion 200 of the metamaterial sensing platform 1 according to one embodiment of the present invention, and FIG. 3A and FIG. 3B are conceptual views schematically illustrating states made before and after the particle T to be sensed is captured in the particle capture portion 200 of the metamaterial sensing platform according to one embodiment of the present invention.

As illustrated in FIGS. 2 and 3, the particle capture portion 200 may have a layered structure with a plurality of slits 201 formed through which the particle T to be sensed is captured.

Specifically, the particle capture portion 200 is configured as a conductor layer formed on the base substrate 100.

The conductive layer may be configured with a metal selected from the group consisting of copper, gold, silver, platinum, and palladium; an alloy or composite containing one or more metals selected from the group consisting of copper, gold, silver, platinum, and palladium and one or more materials selected from the group consisting of graphite, tellurium, tungsten, zinc, iridium, ruthenium, arsenic, phosphorus, aluminum, manganese, and silicon, but is not limited thereto.

In one embodiment of the present invention, Au is used as the material of the particle capture portion 200, and a SiO₂ layer 101 is disposed as an insulating layer between the base substrate 100 and the particle capture portion 200, but the insulating layer may be formed using a non-conductive material having an insulating characteristic without limitation.

For example, the insulating layer may be formed of a metal oxide such as SiO₂, Nb₂O₅, TiO₂, Al₂O₃, or MgO, or a polymer such as polyvinylpyrrolidone (PVP), but is not limited thereto.

In one embodiment of the present invention, a Si layer, which is the base substrate 100, is partially oxidized to form the SiO₂ layer, which gives the surface characteristics such that the surface of the SiO₂ layer may have reactivity to allow the attachment of chemical functional groups.

Meanwhile, the particle capture portion 200 includes a plurality of slits 201 that are patterned with negative engravings such that the surface of the SiO₂ layer 101 disposed on an upper portion of the base substrate 100 is exposed.

Specifically, the plurality of slits 201 may be empty space portions etched into an Au layer in the form of square columns, and the plurality of slits 201 may be formed in the same pattern.

As illustrated in FIG. 2, the plurality of slits S may be formed by fabricating a large amount of recessed patterns on a large area at wafer scale through a photolithography method so that a plurality of holes with predetermined widths and lengths may be formed in the particle capture portion 200 in the form of a matrix with predetermined spacing.

The plurality of slits 201 may focus the terahertz electromagnetic wave on a predetermined area relative to the wavelength beyond the wavelength limit. That is, the terahertz electromagnetic wave may be focused and amplified by the plurality of slits 201 to increase the measurement efficiency on trace particles, and the frequency and concentrated depth of the terahertz electromagnetic wave is adjustable according to the width, thickness, and length of the slits 201.

In one embodiment of the present invention, a 150 nm gold thin film layer was etched to form the slit 201. The slit 201 is 500 nm wide, and was etched such that the surface of the SiO₂ layer 101 of the base substrate 100 is exposed.

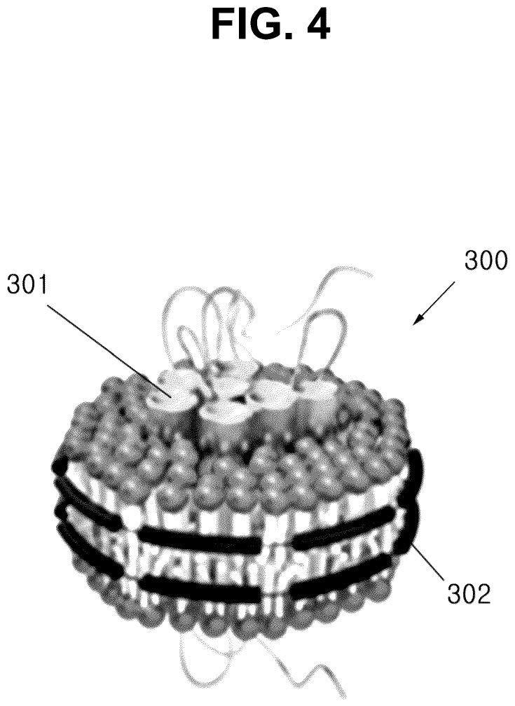

FIG. 4 is a conceptual view schematically illustrating the nanodisk 300 of the metamaterial sensing platform 1 according to one embodiment of the present invention.

As illustrated in FIG. 4, the nanodisk 300 according to one embodiment of the present invention may be assembled by mixing G protein-coupled receptors (GPCRs) 301 produced by E. coli cells with membrane support proteins 302 produced by E. coli cells.

Since the nanodisk 300 is formed as a structure in which lipid membranes are tightly wrapped with the membrane support proteins 302 in the form of the GPCRs 301 reconstituted in the lipid membranes, the nanodisk 300 may have high stability when applied to biosensors.

Meanwhile, in one embodiment of the present invention, receptors involved in depression were expressed and purified using an E. coli system, and MSP proteins were introduced for maintaining the shape of the nanodisk through induction of cell membrane restructuring and refolding for structural stability of GPCR family receptors, which are cell membrane proteins.

Specifically, the nanodisk 300, which includes the GPCRs in one embodiment of the present invention, may be manufactured through steps of: a) producing and purifying the GPCRs from E. coli cells; b) producing and purifying the membrane support proteins from E. coli cells; and c) mixing and stirring the lipids, membrane support proteins, and GPCRs in the sequence of lipids, membrane support proteins, and GPCRs to assemble the nanodisk 300.

In addition, the step of producing and purifying the GPCRs in E. coli cells may include a step of expressing the GPCRs in the form of an inclusion body in the E. coli cells, then lysing the E. coli to release the expressed protein in the form of the inclusion body out of the cells, then dissociating the protein in the form of the inclusion body by mixing the protein with a surfactant and the like, purifying and then restructuring the dissociated protein back into the form of the GPCRs.

Meanwhile, the membrane support protein 302 may use any protein that is added to wrap around the lipid-receptor complex, preferably using a membrane scaffold protein (MSP 1E3D1).

In addition, the lipid may a mixture of one or two or more species selected from 1,2-dipalmitoyl-sn-glycero-3-phosphocholine (DPPC), 1,2-distearoyl-sn-glycero-3-phosphocholine (DSPC), L-a-phosphatidylcholine (HSPC), 1-palmitoyl-2-glutaroyl-sn-glycero-3-phosphocholine (PGPC), 1,2-dilauroyl-sn-glycero-3-phosphocholine (DLPC), 1,2-dimyristoyl-sn-glycero-3-phosphocholine (DMPC), and 1,2-dioleoyl-sn-glycero-3-phosphocholine (DOPC).

It has been confirmed that the nanodisk 300 according to one embodiment of the present invention is activated to interact with the small molecular material that is the particle to be sensed. For example, the nanodisk 300 has stability even in the gas phase, which enables easy introduction into different sensor platforms and may increase the reactivity and affinity for the particle to be sensed as a platform that mimics cell membranes and receptors.

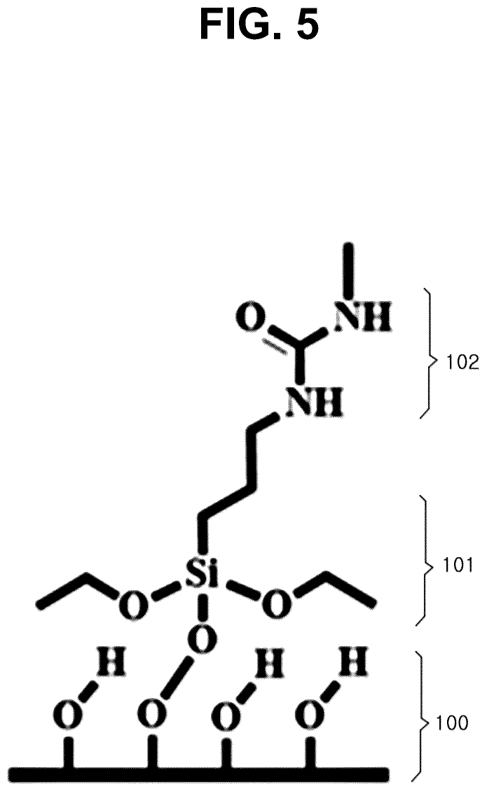

FIG. 5 is a chemical formula schematically illustrating the bonding of an amine group 102 to the SiO₂ layer 101 of the metamaterial sensing platform 1 according to one embodiment of the present invention.

As illustrated in FIG. 5, in one embodiment of the present invention, the amine group (—NH₂) may be introduced through chemical surface treatment of the SiO₂ surface on which the slit 201 is positioned to selectively attach the nanodisk 300 only to a portion where terahertz signal amplification occurs.

Specifically, in one embodiment of the present invention, the interior of the slit 201 is treated with 3-aminopropyltriethoxysilane (APTES), which is a material that readily reacts with the surface of SiO₂, to introduce the amine group, so that a bond may be generated between the amine group on the surface of SiO₂ 101 and the nanodisk 300 through an EDC/NHS reaction.

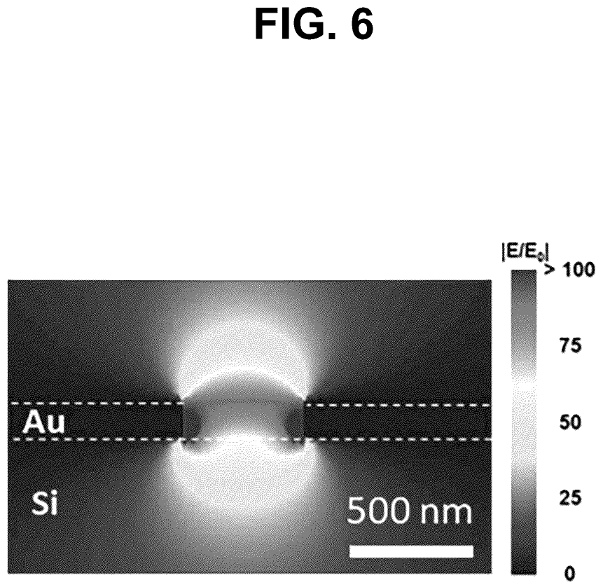

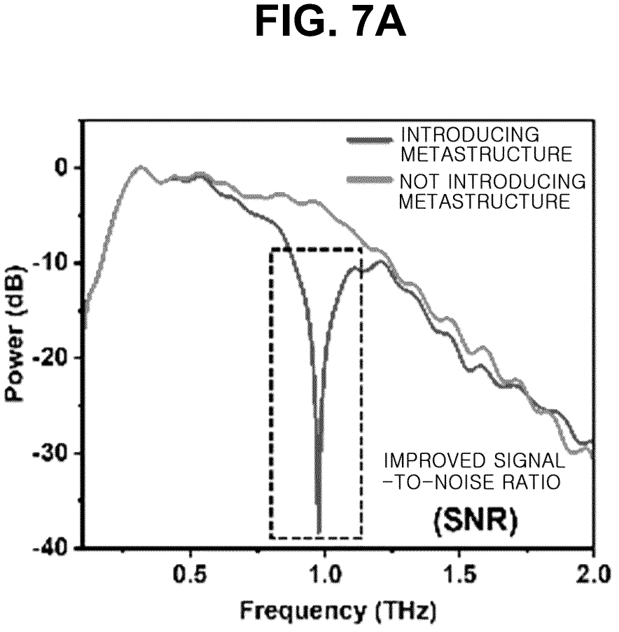

FIG. 6 is a view schematically illustrating the intensity of an electric field concentrated at the slit 201 in the particle capture portion 200 using the metamaterial sensing platform 1 according to one embodiment of the present invention.

As illustrated in FIG. 6, it has been confirmed that the electric field is concentrated in a high density between the Au layer of 150 nm thickness as a result of calculating in simulation the intensity of an electric field concentrated in the slit 201 with Au used as the particle capture portion 200 using the metamaterial sensing platform 1 according to one embodiment of the present invention.

In addition, it has been confirmed that the nanodisk 300 introduced into the slit 201 may be introduced with a height of approximately 30 nm from the surface of the SiO₂ 101, and that the particle to be sensed can be detected with high sensitivity by selectively positioning the nanodisk 300 within a hotspot where the electric field amplification is maximized.

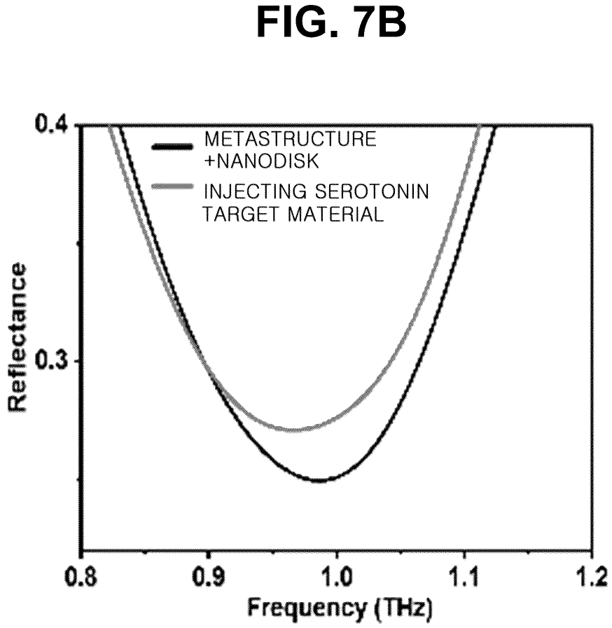

FIG. 7A and FIG. 7B are graphs illustrating a change rate of a signal detected using the metamaterial sensing platform 1 according to one embodiment of the present invention.

As illustrated in FIG. 7A and FIG. 7B, the metamaterial sensing platform 1 according to one embodiment of the present invention may efficiently concentrate the electric field of terahertz wave and amplify the signal, thereby using spectroscopic techniques based on terahertz time.

Specifically, it has been confirmed that a terahertz pulse was output using a laser having a wavelength of 800 nm, incident on the metamaterial sensing platform 1 according to one embodiment of the present invention, and then measured using the reflection signal detected by the detector 22, resulting in a high efficiency in terms of signal-to-noise ratio (SNR). Therefore, it was possible to sensitively analyze whether a serotonin molecule, which is a small molecular material attached to the nanodisk 300, is present.

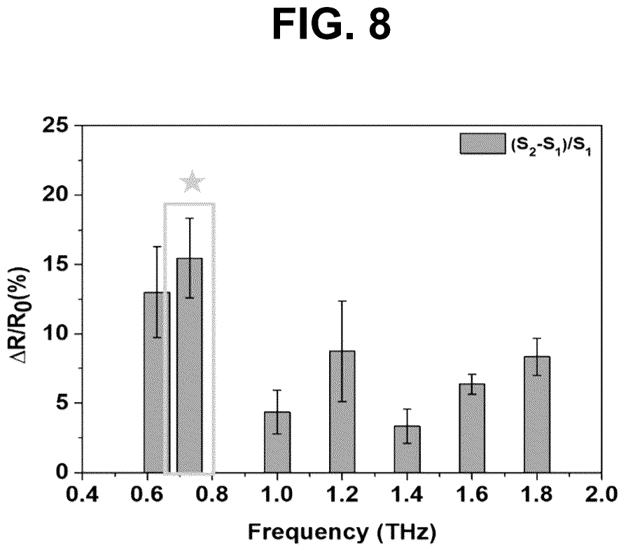

FIG. 8 is a graph illustrating an amplification ratio of a detecting signal using the metamaterial sensing platform 1 according to one embodiment of the present invention.

As illustrated in FIG. 8, with a result of measuring the terahertz reflection signal for a serotonin hormone biomarker using the metamaterial sensing platform 1 according to one embodiment of the present invention, it was observed that the amplification of a signal by a biomarker to be sensed was maximized and a signal change rate was high around 0.72 THz.

That is, it has been confirmed that the signal of the particle T to be sensed that is captured may be maximized using the characteristics of the metamaterial that can modulate a target frequency, thereby enabling the capturing and detection of the biomarker of small molecular material.

A person skilled in the art may understand that the present invention may be carried out in other specific forms with reference to the above-mentioned descriptions without changing the technical spirit or the essential characteristics of the present invention.

Accordingly, it should be understood that the aforementioned embodiments are described for illustration in all aspects and are not limited, and the scope of the present invention shall be represented by the claims to be described below, and it should be construed that all of the changes or modified forms derived from the meaning and the scope of the claims, and an equivalent concept thereto are included in the scope of the present invention.

What is claimed is:

1. A metamaterial sensing platform comprising: a sensing portion configured to capture a particle; and a terahertz sensor portion configured to sense the particle by allowing a terahertz electromagnetic wave to be incident on the sensing portion; wherein the sensing portion includes a base substrate and a particle capture portion disposed on the base substrate and having a plurality of slits formed therein through which the particle is captured, and wherein a nanodisk reactive with the particle is disposed in the plurality of slits.

2. The metamaterial sensing platform of claim 1, wherein the base substrate is a Si layer, and a SiO₂ layer that oxidizes the Si layer is further disposed between the Si layer and the particle capture portion.

3. The metamaterial sensing platform of claim 2, wherein an amine group is interposed in the nanodisk and the nanodisk is attached to the SiO₂ layer.

4. The metamaterial sensing platform of claim 3, wherein the particle capture portion is an Au layer, and the Au layer has a thickness of 150 nm.

5. The metamaterial sensing platform of claim 3, wherein the nanodisk is disposed at a height of 30 nm or less from a surface of the SiO₂.

6. The metamaterial sensing platform of claim 3, wherein the nanodisk includes G protein-coupled receptors (GPCRs).

7. The metamaterial sensing platform of claim 6, wherein the nanodisk is assembled by mixing the G protein-coupled receptors produced from E. coli cells with a membrane support protein produced from E. coli cells.

8. The metamaterial sensing platform of claim 2, wherein the plurality of slits are formed by recessedly etching the particle capture portion such that a surface of the SiO₂ layer is exposed.

9. The metamaterial sensing platform of claim 8, wherein the etching is performed by a photolithography method.

10. The metamaterial sensing platform of claim 1, wherein the terahertz sensor portion senses the particle by allowing the terahertz electromagnetic wave to be incident on the sensing portion and detecting the terahertz electromagnetic wave reflected from the sensing portion.

11. The metamaterial sensing platform of claim 10, wherein the particle is sensed by measuring reflectivity and frequency shift between the terahertz electromagnetic wave being incident on the sensing portion and the terahertz electromagnetic wave being reflected from the sensing portion.
